(19) United States
(12) Patent Application Publication
(10) Pub. No.: US 2018/0273484 A1
(43) Pub. Date: Sep. 27, 2018

(54) DIAZIRINES AS PHOTOCAGES WHICH RELEASE MOLECULES CONTAINING DOUBLE BONDS

(71) Applicant: HENKEL AG & CO. KGAA, Duesseldorf (DE)

(72) Inventors: Thomas Gerke, Duesseldorf (DE); Christian Kropf, Hilden (DE); Ursula Huchel, Cologne (DE); Axel Griesbeck, Cologne (DE); Bjoern Porschen, Cologne (DE)

(21) Appl. No.: 15/761,151

(22) PCT Filed: Aug. 25, 2016
(86) PCT No.: PCT/EP2016/070065

§ 371 Date: Mar. 19, 2018

(30) Foreign Application Priority Data
Sep. 18, 2015 (DE) 10 2015 217 978.8

(51) Int. Cl. C07D 229/02; A61Q 19/10; A61Q 13/00; A61K 8/49; C11D 3/50; A61L 9/01; C11B 9/00

(52) U.S. Cl. CPC C07D 229/02; A61Q 19/10; A61Q 13/00; C11B 9/0092; C11D 3/507; A61L 9/01; A61K 8/494

(57) ABSTRACT

The present invention relates to the field of fragrance storage means as used, for example, in the field of washing or cleaning compositions, cosmetic compositions and air freshening compositions. The invention relates to specific diazirines which function as thermally labile and acid-labile fragrance storage means. The present invention further relates to washing and cleaning compositions, cosmetic compositions and air freshening compositions comprising such diazirines. The invention further relates to a method of long-lasting fragrancing of surfaces, and likewise to a method of long-lasting room fragrancing.

### CROSS-REFERENCE TO RELATED APPLICATION

The present application is a national stage entry according to 35 U.S.C. § 371 of PCT Application No. PCT/EP2016/070065 filed on Aug. 25, 2016, which claims priority to German Patent Application No. 10 2015 217 978.8, filed on Sep. 18, 2015; both of which are herein incorporated by reference in their entirety.

### TECHNICAL FIELD

The subject matter herein generally relates to the field of pro-fragrances, as used, for example, in the field of washing or cleaning agents, cosmetic agents, printer inks, adhesives and air-care agents; and more specifically to diazirines that function as labile pro-fragrances.

### BACKGROUND

Washing and cleaning agents and cosmetic agents generally contain fragrances that impart a pleasant odor to the agents. The fragrances mask the odor of other ingredients, thus giving the user a pleasant impression in terms of odor.

Fragrances, in particular in the field of washing agents, are important components in the composition of washing agents, since it is intended for both damp laundry and dry laundry to have a fresh, pleasant fragrance. In general, fragrances are highly volatile substances, and therefore it is difficult to produce a long-lasting effect of fragrance. In particular in the case of fragrances that produce the fresh, light notes of the perfume and evaporate particularly quickly due to their relatively high vapor pressure, it is difficult to achieve the desired long-lasting impression of fragrance. Pro-fragrance molecules are one option for releasing fragrances in a delayed manner. Depending on the particular pro-fragrance molecule, a covalent bond in the pro-fragrance molecule is broken by the effect of heat, for example, or by reacting with chemical substances, such as acid, and a fragrance is released. Therefore, there is a need for pro-fragrance molecules that can release fragrances by means of a different mechanism.

### SUMMARY

According to a non-limiting embodiment, a compound having formula (I) is presented, i.e.:

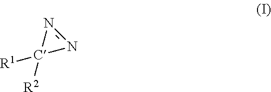

where R¹ and/or R² each have, independently of one another, an abstractable H atom in the alpha position in relation to C′.

According to another non-limiting embodiment, a composition may include the compound where the compound may be, but is not limited to, a washing agent, a cleaning agent, an air-care agent, a cosmetic agent, and combinations thereof.

According to yet another non-limiting embodiment of a method, a method may include applying at least one compound of Formula (I) to at least one surface to be fragranced, and then exposing the surface(s) to light.

### DETAILED DESCRIPTION

It has surprisingly been found that certain diazirines release the stored fragrances when exposed to light. This property is attributed to said diazirines being labile when exposed to light and it thus being possible to release the stored fragrances.

For example, when the compound is used in a laundry treatment agent, such as a washing agent or softener, it was possible to achieve an improved long-term fragrance effect for the treated laundry. The agents also allow the total amount of fragrance contained in the agent to be reduced while still achieving fragrance benefits on the laundered textiles, in particular with regard to the feeling of freshness.

A first embodiment relates to compounds of formula (I)

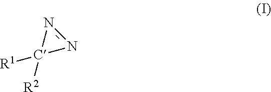

wherein R¹ and/or R² each have, independently of one another, an abstractable H atom in the alpha position in relation to C′.

It has surprisingly been demonstrated that N₂ is split off upon exposure to light, for example day light or even artificial light and in particular light having a wavelength in the range of from 100 nm to 1,000 nm, in particular from 150 nm to 700 nm, alternatively from 200 to 500 nm. This results in the formation of a double bond, and a fragrance molecule, in particular a fragrance alkene, is released. The compound of formula (I) is therefore a novel pro-fragrance.

Further non-limiting embodiments are a method for producing the pro-fragrances of formula (I) and the use of a diazirine of formula (I) as a pro-fragrance.

Yet a further non-limiting embodiment is a washing or cleaning agent comprising at least one compound of formula (I).

A further non-limiting embodiment is a cosmetic agent comprising at least one compound of formula (I).

A further non-limiting embodiment is an air-care agent comprising at least one of the compounds of formula (I).

Yet a further non-limiting embodiment is a method for fragrancing surfaces.

“At least one”, as used herein, refers to one or more, for example 2, 3, 4, 5, 6, 7, 8, 9 or more. In connection with components of the compound described herein, this statement refers not to the absolute quantity of molecules, but rather to the type of component. “At least one compound of formula (I)” therefore means, for example, one or more different compounds of formula (I), i.e. one or more different types of compounds. Together with stated amounts, the stated amounts refer to the total amount of the correspondingly designated type of component, as defined above.

In the present application, “alkyl” refers, in the following, to a saturated aliphatic hydrocarbon, including straight-chain and branched-chain groups. The alkyl group has 1 to 10 carbon atoms; when a numerical range, e.g. “1 to 10”, is stated herein, this means that this group, in the present case the alkyl group, may have 1 carbon atom, 2 carbon atoms, 3 carbon atoms, etc., up to and including 10 carbon atoms. In particular, the alkyl may be a medium alkyl, having 1 to 6 carbon atoms, or a lower alkyl, having 1 to 4 carbon atoms, for example methyl, ethyl, n-propyl, isopropyl, butyl, isobutyl, tert-butyl, etc.

“Alkenyl” refers to an alkyl group, as defined herein, made up of at least two carbon atoms and at least one carbon-to-carbon double bond, for example ethenyl, propenyl, butenyl or pentenyl and structural isomers thereof, such as 1- or 2-propenyl, 1-, 2-, or 3-butenyl, etc.

“Alkynyl” refers to an alkyl group, as defined herein, made up of at least two carbon atoms and at least one carbon-to-carbon triple bond, for example ethynyl (acetylene), propynyl, butynyl or pentynyl and structural isomers thereof as described above.

“Heteroalkyl”, “heteroalkenyl” and “heteroalkynyl”, as used herein, refer to alkyl, alkenyl and alkynyl groups, respectively, as defined above, in which one or more carbon atoms are replaced by heteroatoms, in particular selected from O, S, N and Si, for example ethoxyethyl, ethoxyethenyl, isopentoxypropyl, etc.

A “cycloalkyl” group refers to monocyclic, dicyclic or polycyclic groups, in particular made up of 3 to 8 carbon atoms, in which the ring is made up of carbon atoms that are interconnected by carbon-to-carbon single bonds, carbon-to-carbon double bonds and/or carbon-to-carbon triple bonds. The ring may comprise one, two or more or no double and/or triple bonds. However, said ring does not have a complete conjugated pi electron system. For example, a cycloalkyl functional group is cyclopropyl, cyclobutyl, cyclopentyl, cyclohexyl, cyclobutenyl, cyclopentenyl, cyclohexenyl, etc. Examples of cycloalkyl groups are cyclopropane, cyclobutane, cyclopentane, cyclopentene, cyclohexane, adamantane, cyclohexadiene, cycloheptane and cycloheptatriene.

“Aryl” refers to monocyclic or polycyclic, i.e. rings having shared neighboring carbon atom pairs, groups made up in particular of 6 to 14 carbon ring atoms that have a completely conjugated pi electron system. Examples of aryl groups are phenyl, naphthalenyl and anthracenyl.

A “heteroaryl” group refers to monocyclic, dicyclic or polycyclic rings that share a neighboring ring atom pair, aromatic rings made up in particular of 5 to 10 ring atoms, with one, two, three or four ring atoms being nitrogen, oxygen or sulfur, and the remainder being carbon. Examples of heteroaryl groups are pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, isobenzofuryl, benzothienyl, benzotriazolyl, isobenzothienyl, indolyl, isoindolyl, 3H-indolyl, benzimidazolyl, benzothiazolyl, benzoxazolyl, quinolizinyl, quinazolinyl, pthalazinyl, quinoxalinyl, cinnolinyl, naphthyridinyl, quinolyl, isoquinolyl, tetrazolyl, 5,6,7,8-tetrahydroquinolyl, 5,6,7,8-tetrahydroisoquinolyl, purinyl, pteridinyl, pyridinyl, pyrimidinyl, carbazolyl, xanthenyl or benzoquinolyl.

A “heterocycloalkyl” group refers to a monocyclic or fused ring made up of 5 to 10 ring atoms, containing one, two or three heteroatoms selected from N, O and S, the remainder of the ring atoms being carbon atoms. A “heterocycloalkenyl” group additionally contains one or more double bonds. However, the ring does not have a complete conjugated pi electron system. Examples of heteroalicyclic groups are pyrrolidine, piperidine, piperazine, morpholine, imidazolidine, tetrahydropyridazine, tetrahydrofuran, thiomorpholine, tetrahydropyridine, and the like.

The compound of the present non-limiting embodiment has the general formula (I)

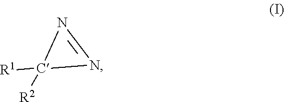

wherein R¹ and/or R² each having, independently of one another, an abstractable H atom in the alpha position in relation to C′.

According to Formula (I), R¹ and R² may be each selected, independently of one another, from

a linear, aliphatic, olefinic or open-chain organic group having 2 to 20 carbon atoms, alternatively from 2 to 12 carbon atoms, and having 0 to 10 heteroatoms selected from N, O, S and Si; or
a branched or cyclic organic group having 3 to 20 carbon atoms, alternatively from 3 to 12 carbon atoms, and having 0 to 10 heteroatoms selected from N, O, S and Si; or
an aromatic or heteroaromatic organic group having 4 to 20 carbon atoms, alternatively from 4 to 12 carbon atoms, and having 0 to 10 heteroatoms selected from N, O, S and Si; or
R¹ and R² are interconnected by a bridge Q for ring closure and form a bridging part —R¹-Q-R²—, R¹ and R² each representing, independently of one another, a group having 1 to 5 carbon atoms; and Q representing a substituted or unsubstituted bridging group having 1 or 2 to 20 carbon atoms, alternatively from 2 to 12 carbon atoms, and having 0 to 10 heteroatoms selected from N, O, S and Si.

R¹ and/or R² each have, independently of one another, an abstractable H atom in the alpha position in relation to C′. If therefore R¹, for example, has an abstractable H atom in the alpha position, the structure (Ia) shown below results; if R² has an abstractable H atom in the alpha position in relation to C′, the structure (Ib) shown below results; if both R¹ and R² have a corresponding H atom, the structure (Ic) results.

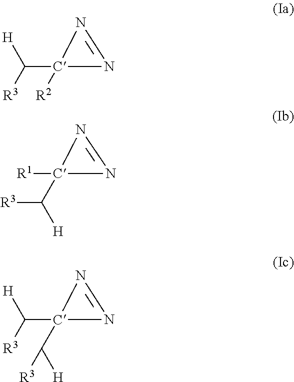

In non-limiting embodiments, R¹ and/or R² are therefore each, independently of one another,
a. a functional group of formula —CH(OH)—R³, —CH₂—C(O)—R³, aryl, heteroaryl, CH₂ aryl or CH₂ heteroaryl, wherein R³ being selected from the group consisting of hydrogen, substituted or unsubstituted, linear or branched alkyl, alkenyl or alkynyl having up to 20, alternatively up to 10, carbon atoms, substituted or unsubstituted, linear or branched heteroalkyl, heteroalkenyl or heteroalkynyl having up to 20, alternatively up to 10, carbon atoms, and having 1 to 6, alternatively 1 to 4, heteroatoms selected from O, S and N, substituted or unsubstituted aryl having up to 20, alternatively 6 to 10, carbon atoms, substituted or unsubstituted heteroaryl having up to 20, alternatively 4 to 10, carbon atoms, and having 1 to 6, alternatively 1 to 4, heteroatoms selected from O, S and N, cycloalkyl or cycloalkenyl having up to 20, alternatively 5 to 10, carbon atoms, and heterocycloalkyl or heterocycloalkenyl having up to 20, alternatively 4 to 10, carbon atoms, and having 1 to 6, alternatively 1 to 4, heteroatoms selected from O, S and N; or
b. a substituted or unsubstituted, linear alkyl having 1 to 5 carbon atoms, or a substituted or unsubstituted, branched alkyl having 3 to 5 carbon atoms.

As used herein, “substituted” means that one or more hydrogen atoms, methyl groups, methylene groups, methine groups or quaternary carbon atoms of the functional groups R¹, R² or R³ of the pro-fragrance may each, independently of one another, be substituted by heteroatoms. Heteroatoms as used herein are selected from the group of nitrogen, oxygen, sulfur, silicon, selenium, phosphorus, fluorine, chlorine, bromine or iodine. One or more hydrogen atoms or methyl groups may be substituted by a heteroatom selected from the group comprising nitrogen, oxygen, sulfur, silicon, selenium, phosphorus, fluorine, chlorine, bromine or iodine, one or more methylene groups may be substituted by a heteroatom selected from the group of nitrogen, oxygen, sulfur, selenium, phosphorus or silicon, one or more methine groups may substituted by a heteroatom selected from the group of nitrogen, phosphorus or silicon, and one or more quaternary carbon atoms may be substituted by silicon. If free valencies arise as a result of the substitution of a hydrogen atom, a methyl group, a methylene group or a methine group, these are in principle saturated with hydrogen. A terminal methyl group adjacent to a methylene group may therefore, for example, be exchanged for a hydroxy group or a sulfanyl group such that a methylene hydroxy group or a methylene thiol group is obtained. Similarly, an isopropyl group that is a group having two methyl groups and a methine group, or a derivative of the isopropyl group that is a group having a methyl group, a methylene group and a quaternary carbon atom, may have, for example, the following substitution patterns:

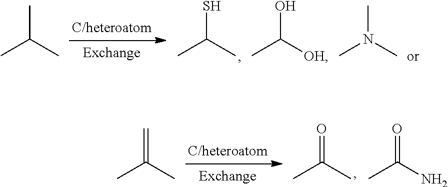

Hydrogen atoms, methyl groups, methylene groups, methine groups or quaternary carbon atoms of the groups R¹, R² or R³ may in principle be substituted as desired by heteroatoms, however, no two directly adjacent groups are substituted by heteroatoms at the same time, with the exception of disulfides or polysulfides.

It is also possible for R¹ and R² to be interconnected by a bridge element Q for ring closure. In this case, the compound may have the following formula (II).

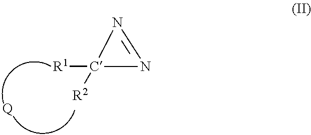

The R¹ and R² in the bridging part —R¹-Q-R²— may represent, independently of one another, a group having 1 to 5 carbon atoms, wherein Q representing a substituted or unsubstituted bridging group having 1 or 2 to 20 carbon atoms, in particular 2 to 12 carbon atoms, and having 0 to 10 heteroatoms selected from N, O, S and Si. R¹ and R² may represent, independently of one another, a group having 1 to 5 carbon atoms, wherein at least one atom of the groups R¹ and R² being a secondary, tertiary or quaternary C atom in the alpha position in relation to C′, and Q representing a substituted or unsubstituted group having 1 to 10 carbon atoms that bridges R¹ and R².

In a non-limiting embodiment, R¹ and R² in formula (II) represent, independently of one another, a group having 1 to 5 carbon atoms, wherein at least one atom of the groups R¹ and R² being a secondary or tertiary carbon atom in the alpha position in relation to C′. In another non-limiting embodiment, one of the two groups R¹ and R² represents a group having 1 to 3 carbon atoms, wherein at least one atom of the groups R¹ and R² being a secondary carbon atom in the alpha position in relation to C′, while the other group R¹ or R² represents a group having 1 to 3 carbon atoms, wherein at least one atom of the groups R¹ and R² being a tertiary carbon atom in the alpha position in relation to C′.

In another non-limiting embodiment, Q represents a substituted or unsubstituted group that bridges R¹ and R², wherein the part of Q that bridges R¹ and R² being selected such that a four-membered, five-membered, six-membered, seven-membered or eight-membered ring is present. Q is may be a hydrocarbon.

The bridging part —R¹-Q-R²— may be a hydrocarbon.

The group R¹ and/or the group R² or the bridging part R¹-Q-R² may be a group derived from a fragrance alkene. “Derived group” refers to the group that arises when, formally, the unsaturated group of a fragrance alkene bonds to nitrogen and one of the two carbon atoms of the double bond of the alkene forms the bond to the nitrogen, as shown in formula (I).

In a non-limiting embodiment, the fragrance alkene contains a semicyclic or exocyclic double bond if R¹ or R² are cyclic compounds or R¹ is connected to R² by Q for ring closure. As used herein, the term “endocyclic double bond”/“cyclic double bond” is understood to mean a double bond in which both connected atoms are ring atoms. As used herein, the term “exocyclic double bond” is understood to mean a double bond in which neither of the two atoms connected by the double bond is a ring atom. As used herein, the term “semicyclic double bond” is understood to mean a double bond in which one of the two atoms interconnected by the double bond is a ring atom and the other atom lies outside the ring:

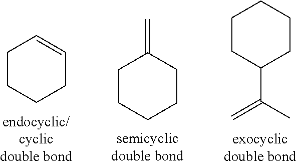

The diazirine can be formed by means of said double bond. Upon exposure to light, nitrogen is split off, thus releasing the fragrance.

The exposure may be to natural light or to light that is generated by common domestic lighting, in particular light having a wavelength in the range of from 100 nm to 1,000 nm, in particular from 150 nm to 700 nm, alternatively from 200 nm to 500 nm. Exposure lasting a few minutes is enough to release the fragrances.

If Q is substituted, Q is substituted by one or more groups which, independently of one another, are bonded to Q by C and/or N atoms optionally contained in the group, alternatively by C atoms contained in the group. If Q is directly bonded to a C atom contained in the group, the group is a straight-chain or branched, saturated or unsaturated hydrocarbon group having up to 6 carbon atoms. If Q is substituted, Q may be substituted by an isopropenyl group.

If R¹ and/or R² and/or Q are substituted, stereocenters may arise as a result of the substitutions. Stereocenters of this kind may have the (R) configuration, the (S) configuration and mixtures of the (R) configuration and the (S) configuration.

In a non-limiting embodiment, the pro-fragrance may be derived from limonene and has the following structure (III):

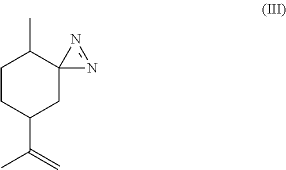

The pro-fragrance molecule may be present in various configurations, as shown by the following formulae (IIIa) and (IIIb) by way of example:

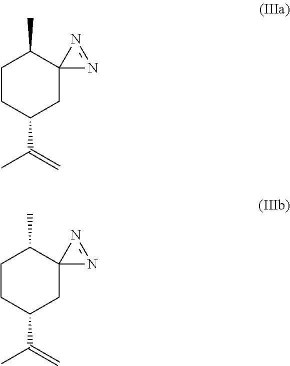

If nitrogen (N₂) splits off from said pro-fragrances, limonene is released as an odorant. In said non-limiting embodiment, limonene is the fragrance alkene from which the pro-fragrance is derived. Limonene is one of the most important fragrances in the field of washing and cleaning agents. The odor of limonene is often associated with freshness, which is synonymous with cleanliness and purity for the user.

The pro-fragrance may be derived from common fragrances that have an unsaturated C-to-C bond, i.e. a double bond or a triple bond. Suitable fragrance alkenes are, for example, benzyl cinnamate, cinnamyl alcohol, ambrettolide, isoeugenol acetate, hexenyl benzoate, phenylethyl cinnamate, aldehyde C11 enic, amyl cinnamaldehyde, dipentene, cinnamaldehyde, undecylene, 2-hexenol, dihydromyrcenol, hexyl cinnamaldehyde, acedyl, propidyl, 9-decenol, citral diethyl acetal, citrusal, geranial, neral, lyral, allyl ionone, hexenyl salicylate, allyl amyl glycolate, brahmanol, phenyl acetate, citronellol, citronellyl acetate, cyclomethylene citronellol, cinnamyl acetate, linalool, linalyl formate, linalyl acetate, jasmone, undecavertol, myrcenyl acetate, bacdanol, nerolidol, allyl hexanoate, allyl heptanoate, ethyl linalool, epitone, gyrane, neolavandate, iso E super, precyclemone, polysantol, benzylideneacetone, isoeugenol, eugenol, geraniol, triplal, allyl phenylacetate, 3-dodecenal, alpha-methyl ionone, alpha-ionone, beta-ionone, farenal, sandalore, tangerinol, karanal, dihydro isojasmone, leguminal, diantheme, hexenyl capronate, hexenyl isovalerate, neryl acetate, maltol, cyclopidene, citronellal, ebanol, vetiveryl acetate, farnesol, butyl cinnamaldehyde, rose oxide, isopentyrate, dupical, vernaldehyde, isobutyl angelate, isobutyl tiglate, melonal, ambrinol, dynascone, koavone, nerol oxide, nectaryl, anethol, spirofluorine, cyclovertal, fleuroxene, vertosine, myrcene, isopulegol, gamma-terpinene, 4-terpinen-1-ol, ocimene, valencene, gamma-cadinene, delta-carene, alpha, cedrene, carveol, farnesene, perillaldehyde, myrtenal, myrtenol, germacrene, citronellyl formate, geranyl formate, citronellyl propionate, geranyl butyrate, geranyl propionate, carveol, propyl angelate, alpha-pinene, alpha-terpinene, limonene, beta-bisabolene, humulene, terpinolene, phellandrene, trichodiene, beta-asarone, alpha-thujene, beta-methyl ionone, vetiverol, beta-vetivene, gamma-vetivene, methyl jasmonate, methyl cinnamaldehyde, 2,6-nonadienal, sandalmysore, gergamal, alpha, patchoulene, dihydrolinalool, claritone, prenyl isobutyrate, terrestral, liminal, octalynol, azarbre, azuril, bigarade oxide, dimethyl octanone, givescone, isocyclocitral, geranyl ethyl ether, isoamyl cinnamate, dihydrolinalyl acetate, isofreshal, floral super, terpinyl methyl ether, greenyl isobutyrate, and in particular limonene, alpha-phellandrene, beta-phellandrene, alpha-pinene, beta-pinene, camphene, caryophyllene, longifolene, ocimene, alpha-terpinene, beta-terpinene, gamma-terpinene, alpha-terpineol, delta-terpineol, gamma-terpineol, beta-terpineol, alpha-citronellol, beta-citronellol, alpha-citronellal, beta-citronellal, linalool, geraniol, santalol, hasmigone, carvone, 2-carene, 3-carene, 4-carene, elemol and/or curcumene.

A further aspect of includes a method for producing a compound of general formula (I)

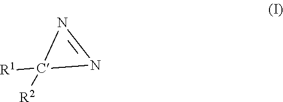

wherein R¹ and/or R² each have, independently of one another, an abstractable H atom in the alpha position in relation to C′, by dehydration of a diaziridine of general formula (IV)

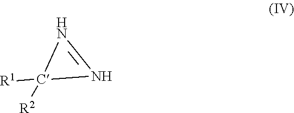

The diaziridines (IV) may be obtained by reacting aldehydes, of general formulae (Va) and (Vb), or ketones, of general formula (Vc).

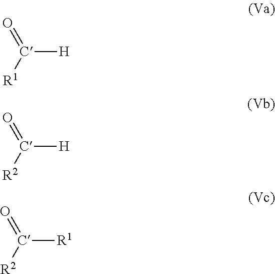

In this case, ketones or aldehydes may be reacted, for example, with ammonia and hydroxylamine sulfonic acid in accordance with known methods. The diaziridines may then be dehydrated by mild oxidants to form diazirines. Suitable oxidants are, for example, chromium trioxide/sulfuric acid, iodine, silver oxide or t-butyl hypochlorite.

In a non-limiting embodiment, a ketone is used as a starting product. The reaction corresponds in this case to the following reaction mechanism:

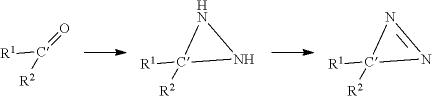

The ketones (Vc) or the aldehydes (Va), (Vb) may be fragrance ketones or fragrance aldehydes, respectively. All common fragrance aldehydes and fragrance ketones that are typically used to bring about a pleasant feeling of fragrance may be used as the fragrance aldehydes or fragrance ketones. Suitable fragrance aldehydes and fragrance ketones are generally known to a person skilled in the art. The fragrance ketones may include all ketones that are able to impart a desired fragrance or feeling of freshness. It is also possible to use mixtures of various ketones. Ketones that can be used are, for example, alpha-damascone, delta-damascone, iso-damascone, carvone, gamma-methyl ionone, iso E super, 2,4,4,7-tetramethyl-oct-6-en-3-one, benzyl acetone, beta-damascone, damascenone, methyl dihydrojasmonate, methyl cedrylone, hedione and mixtures thereof. Aldehydes may be any desired aldehydes which, in the manner of fragrance ketones, impart a desired fragrance or a feeling of freshness. The aldehydes may be individual aldehydes or mixtures of aldehydes. Suitable aldehydes are, for example, melonal, triplal, ligustral, adoxal, lilial and so on. The fragrance aldehydes and fragrance ketones may have an aliphatic, cycloaliphatic, aromatic, ethylenically unsaturated structure or a combination of said structures. Further heteroatoms or polycyclic structures may further be present. The structures may comprise suitable substituents, such as hydroxyl or amino groups. Suitable ester-type fragrances are, for example, benzyl acetate, phenoxyethyl isobutyrate, p-tert-butylcyclohexyl acetate and so on. Hydrocarbon-type fragrance compounds are, for example, terpenes such as limonene and pinene. Suitable ether-type fragrances are, for example, benzyl ethyl ether and ambroxane. Suitable fragrance alcohols are, for example, 10-undecen-1-ol, 2,6-dimethylheptan-2-ol, 2-methylbutanol, 2-methylpentanol, 2-phenoxyethanol, 2-phenylpropanol and so on. Fragrances or perfume oils may also be natural fragrance mixtures, as are obtainable from plant sources. The fragrances or perfume oils may also be essential oils, such as angelica root oil, anise oil, arnica blossom oil and so on.

In a non-limiting embodiment, the fragrance ketone of general formula (Vc) is dihydrocarvone (5-isopropenyl-2-methylcyclohexanone). Dihydrocarvone has two stereocenters. The ketone of general formula (Vc) is selected from the group consisting of (2S,5S)-5-isopropenyl-2-methylcyclohexanone ((−)-dihydrocarvone), (2S,5R)-5-isopropenyl-2-methylcyclohexanone ((+)-isodihydrocarvone), (2R,5R)-5-isopropenyl-2-methylcyclohexanone ((+)-dihydrocarvone), (2R,5S)-5-isopropenyl-2-methylcyclohexanone ((−)-isodihydrocarvone), or mixtures thereof. In a non-limiting embodiment, the ketone of general formula (Va) is (2S,5R)-5-isopropenyl-2-methylcyclohexanone ((+)-isodihydrocarvone) or (2R,5R)-5-isopropenyl-2-methylcyclohexanone ((+)-dihydrocarvone), or mixtures thereof.

A pro-fragrance of general formula (I) which, when exposed to light, releases limonene, which is one of the most important fragrances in the field of washing and cleaning agents, is obtained from (+)-dihydrocarvone and/or (+)-isodihydrocarvone in accordance with the method. The odor of limonene is often associated with freshness, which is synonymous with cleanliness and purity for the user.

A further non-limiting aspect relates to the use of diazirines of general formula (I) as pro-fragrances.

A further non-limiting is a washing or cleaning agent, such as a washing agent, softener or auxiliary washing agent, containing at least one compound of formula (I). In various embodiments, said compound is contained in amounts of from 0.001 to 5 wt. %, alternatively from 0.005 to 4 wt. %, more alternatively from 0.005 to 3 wt. %, particularly alternatively from 0.01 to 2 wt. %, in each case based on the overall agent. Suitable cleaning agents are, for example, cleaning agents for hard surfaces, such as dishwasher detergents. The cleaning agents may also be, for example, household cleaners, all-purpose cleaners, window cleaners, floor cleaners, etc. The cleaning agent may be a product for cleaning toilet bowls and urinals, advantageously a flush cleaner for being hung in the toilet bowl.

According to a non-limiting embodiment, the washing or cleaning agent contains at least one surfactant selected from anionic, cationic, nonionic, zwitterionic and amphoteric surfactants or mixtures thereof.

According to a further non-limiting embodiment, the agent is present in solid or liquid form.

A further subject is a cosmetic agent, containing at least one compound of formula (I), that contains the compound in amounts of from 0.001 to 5 wt. %, alternatively from 0.005 to 4 wt. %, more alternatively from 0.005 to 3 wt. %, particularly alternatively from 0.01 to 2 wt. %, in each case based on the overall agent.

A further subject is an air-care agent, for example a room air-freshener, room deodorizer, room spray, etc., containing at least one compound of formula (I), wherein the compound of formula (I) being contained in amounts of from 0.001 to 5 wt. %, alternatively from 0.005 to 4 wt. %, more alternatively from 0.005 to 3 wt. %, particularly alternatively from 0.01 to 2 wt. %, in each case based on the total weight of the agent.

According to a further non-limiting embodiment, additional fragrances are contained in an agent, i.e. a washing or cleaning agent, cosmetic agent or air-care agent, said additional fragrances being in particular selected from the group comprising fragrances of natural or synthetic origin, such as more volatile fragrances, higher-boiling fragrances, solid fragrances and/or adherent fragrances.

Examples of adherent odorants may be or include essential oils such as angelica root oil, anise oil, arnica blossom oil, basil oil, bay oil, bergamot oil, champaca blossom oil, noble fir oil, noble fir cone oil, elemi oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, cajuput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemongrass oil, lime oil, mandarin oil, melissa oil, musk seed oil, myrrh oil, clove oil, neroli oil, niaouli oil, olibanum oil, orange oil, oregano oil, palmarosa oil, patchouli oil, Peru balsam oil, petitgrain oil, pepper oil, peppermint oil, allspice oil, pine oil, rose oil, rosemary oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil and cypress oil.

However, higher-boiling and solid odorants of natural or synthetic origin may be or include, but are not limited to, ambrettolide, alpha-amylcinnamaldehyde, anethole, anisaldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerianate, borneol, bornyl acetate, alpha-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl n-amyl ketone, methylanthranilic acid methyl ester, p-methylacetophenone, methylchavicol, p-methylquinoline, methyl beta-naphthyl ketone, methyl n-nonyl acetaldehyde, methyl n-nonyl ketone, muscone, beta-naphthol ethyl ether, beta-naphthol methyl ether, nerol, nitrobenzene, n-nonyl aldehyde, nonyl alcohol, n-octylaldehyde, p-oxyacetophenone, pentadecanolide, beta-phenethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, gamma-undecalactone, vanillin, veratraldehyde, cinnamaldehyde, cinnamyl alcohol, cinnamic acid, cinnamic acid ethyl ester, cinnamic acid benzyl ester. The more volatile fragrances include in particular lower-boiling odorants of natural or synthetic origin, which may be used alone or in mixtures. Examples of more volatile fragrances are alkyl isothiocyanates (alkyl mustard oils), butanedione, limonene, linalool, linayl acetate and propionate, menthol, menthone, methyl-n-heptenone, phellandrene, phenylacetaldehyde, terpinyl acetate, citral and citronellal.

According to a further non-limiting embodiment, the agent, i.e. a washing or cleaning agent, cosmetic agent or air-care agent, comprises at least one, alternatively a plurality of, active components, in particular washing, care, cleansing and/or cosmetic components, advantageously selected from the group comprising anionic surfactants, cationic surfactants, amphoteric surfactants, nonionic surfactants, acidifying agents, alkalizing agents, anti-crease compounds, antibacterial substances, antioxidants, anti-redeposition agents, antistatic agents, builders, bleaching agents, bleach activators, bleach stabilizers, bleach catalysts, ironing aids, cobuilders, fragrances, shrinkage preventers, electrolytes, enzymes, color protectants, colorants, dyes, dye transfer inhibitors, fluorescing agents, fungicides, germicides, odor-complexing substances, adjuvants, hydrotropes, rinse aids, complexing agents, preservatives, corrosion inhibitors, water-miscible organic solvents, optical brighteners, perfumes, perfume carriers, luster agents, pH adjusters, proofing and impregnation agents, polymers, anti-swelling and anti-slip agents, foam inhibitors, phyllosilicates, soil-repellent substances, silver protectants, silicone oils, soil-release active ingredients, UV protection substances, viscosity regulators, thickeners, discoloration inhibitors, graying inhibitors, vitamins and/or softeners. As used herein, stated amounts in wt. % refer to the total weight of the agent.

The amounts of the individual ingredients in the agents, i.e. a washing or cleaning agent, cosmetic agent or air-care agent, depend on the intended purpose of the agents in question, and a person skilled in the art is in principle familiar with the ranges of the amounts of the ingredients that should be used, or may obtain these from the relevant technical literature. Depending on the intended purpose of the agents, the surfactant content, for example, is selected to be higher or lower. The surfactant content of washing agents may typically be, for example, between 10 and 50 wt. %, alternatively between 12.5 and 30 wt. %, and in particular between 15 and 25 wt. %, while, for example, cleaning agents for automatic dishwashing may contain, for example, between 0.1 and 10 wt. %, alternatively between 0.5 and 7.5 wt. %, and in particular between 1 and 5 wt. % surfactants.

The agents, i.e. a washing or cleaning agent, cosmetic agent or air-care agent, may contain surfactants, such as anionic surfactants, nonionic surfactants and mixtures thereof, but also cationic surfactants. Suitable nonionic surfactants are in particular ethoxylation and/or propoxylation products of alkyl glycosides and/or linear or branched alcohols each having 12 to 18 carbon atoms in the alkyl portion and 3 to 20, alternatively 4 to 10, alkyl ether groups. Also usable are corresponding ethoxylation and/or propoxylation products of N-alkylamines, vicinal diols, fatty acid esters and fatty acid amides which, with regard to the alkyl portion, correspond to the stated long-chain alcohol derivatives, and of alkylphenols having 5 to 12 carbon atoms in the alkyl group.

Suitable anionic surfactants are in particular soaps and those containing sulfate or sulfonate groups having alkali ions as cations. Usable soaps are alkali salts of saturated or unsaturated fatty acids having 12 to 18 carbon atoms. Fatty acids of this kind may also be used in a not completely neutralized form. Usable sulfate-type surfactants include salts of sulfuric acid semiesters of fatty alcohols having 12 to 18 carbon atoms and the sulfation products of the stated nonionic surfactants having a low degree of ethoxylation. Usable sulfonate-type surfactants include linear alkylbenzenesulfonates having 9 to 14 carbon atoms in the alkyl portion, alkanesulfonates having 12 to 18 carbon atoms, and olefin sulfonates having 12 to 18 carbon atoms, resulting from the reaction of corresponding monoolefins with sulfur trioxide, and alpha-sulfo fatty acid esters resulting from the sulfonation of fatty acid methyl or ethyl esters.

Cationic surfactants are selected from among esterquats and/or quaternary ammonium compounds (QAC) of general formula (R^{I})(R^{II})(R^{III})(R^{IV})N⁺X⁻, in which R^{I} to R^{IV} represent C₁₋₂₂ alkyl groups, C₇₋₂₈ arylalkyl groups or heterocyclic groups that are the same or different, where two groups or, in the case of aromatic bonding such as in pyridine, even three groups form, together with the nitrogen atom, the heterocycle, for example a pyridinium or imidazolinium compound, and X⁻ represents halide ions, sulfate ions, hydroxide ions or similar anions. QACs may be produced by reacting tertiary amines with alkalizing agents, such as methyl chloride, benzyl chloride, dimethyl sulfate, dodecyl bromide, but also ethylene oxide. The alkylation of tertiary amines with a long alkyl functional group and two methyl groups is particularly simple; the quaternization of tertiary amines with two long groups and a methyl group may also be carried out under mild conditions using methyl chloride. Amines having three long alkyl groups or hydroxy-substituted alkyl groups are less reactive, and are quaternized using dimethyl sulfate, for example. Examples of suitable QACs are benzalkonium chloride (N-alkyl-N,N-dimethylbenzyl ammonium chloride), Benzalkon B (m,p-dichlorobenzyldimethyl-C₁₂-alkylammonium chloride, benzoxonium chloride (benzyldodecyl-bis-(2-hydroxyethyl) ammonium chloride), cetrimonium bromide (N-hexadecyl-N,N-trimethylammonium bromide), benzethonium chloride (N,N-dimethyl-N-[2-[2-[p-(1,1,3,3-tetramethylbutyl)phenoxy]ethoxy]ethyl]benzylammonium chloride), dialkyldimethyl ammonium chlorides such as di-n-decyldimethyl ammonium chloride, didecyldimethyl ammonium bromide, dioctyldimethyl ammonium chloride, 1-cetylpyridinium chloride and thiazoline iodide, and mixtures thereof. QACs are benzalkonium chlorides having C₈-C₂₂ alkyl groups, in particular C₁₂-C₁₄ alkylbenzyldimethyl ammonium chloride.

Non-limiting esterquats are methyl-N-(2-hydroxyethyl)-N,N-di(talgacyloxyethyl) ammonium methosulfate, bis-(palmitoyl)ethylhydroxyethylmethyl ammonium methosulfate, or methyl-N,N-bis(acyloxyethyl)-N-(2-hydroxyethyl) ammonium methosulfate. Commercially available examples are the methylhydroxyalkyldialkoyloxyalkyl ammonium methosulfates marketed by Stepan under the trademark Stepantex®, the products from BASF SE known under the trade name Dehyquart, or the products from the manufacturer Evonik known under the name Rewoquat.

Surfactants are contained in the agents, i.e. a washing or cleaning agent, cosmetic agent or air-care agent, in amount proportions of from 5 wt. % to 50 wt. %, alternatively from 8 wt. % to 30 wt. %, alternatively up to 30 wt. %, in particular from 5 wt. % to 15 wt. %, surfactants, including cationic surfactants at least in part, as used in particular in laundry aftertreatment agents.

An agent, in particular a washing or cleaning agent, contains at least one water-soluble and/or water-insoluble, organic and/or inorganic builder. The water-soluble organic builders include polycarboxylic acids, in particular citric acid and sugar acids, monomeric and polymeric aminopolycarboxylic acids, in particular methylglycinediacetic acid, nitrilotriacetic acid and ethylenediaminetetraacetic acid, and polyaspartic acid, polyphosphonic acids, in particular amino tris(methylenephosphonic acid), ethylenediamine tetrakis(methylenephosphonic acid) and 1-hydroxyethane-1,1-diphosphonic acid, polymeric hydroxy compounds such as dextrin, and polymeric (poly)carboxylic acids, polymeric acrylic acids, methacrylic acids, maleic acids and mixed polymers thereof, which may also contain, in the polymer, small proportions of polymerizable substances without a carboxylic acid functionality. Compounds of this class which are suitable, although less preferred, are copolymers of acrylic acid or methacrylic acid with vinyl ethers, such as vinyl methyl ethers, vinyl ester, ethylene, propylene and styrene, in which the proportion of the acid is at least 50 wt. %. The organic builders may, in particular for the production of liquid agents, be used in the form of aqueous solutions, in the form of 30 to 50 wt. % aqueous solutions. All indicated acids are generally used in the form of water-soluble salts thereof, in particular alkali salts thereof.

Organic builders may, if desired, be contained in amounts of up to 40 wt. %, in particular up to 25 wt. %, and alternatively from 1 wt. % to 8 wt. %. Amounts close to the stated upper limit are used in paste-form or liquid, in particular water-containing, agents in a non-limiting embodiment. Laundry aftertreatment agents, such as softeners, may optionally also be free of organic builder.

In particular alkali silicates and polyphosphates, such as sodium triphosphate, are suitable as water-soluble inorganic builder materials. In particular crystalline or amorphous alkali aluminosilicates may, if desired, be used in amounts of up to 50 wt. %, alternatively no more than 40 wt. %, and, in liquid agents, in particular from 1 wt. % to 5 wt. %, as water-insoluble, water-dispersible inorganic builder materials. Among these, crystalline sodium aluminosilicates of washing agent quality, in particular zeolite A, P and optionally X, are used. Amounts close to the stated upper limit are preferably used in solid particulate agents. Suitable aluminosilicates have in particular no particles having a particle size greater than 30 μm and alternatively comprise at least 80 wt. % particles having a size smaller than 10 μm.

Suitable substitutes or partial substitutes for the stated aluminosilicate are crystalline alkali silicates, which may be present alone or in a mixture with amorphous silicates. The alkali silicates that are usable in the agents as builders have a molar ratio of alkali oxide to SiO₂ of less than 0.95, in particular from 1:1.1 to 1:12, and may be present in amorphous or crystalline form. Non-limiting alkali silicates are sodium silicates, in particular amorphous sodium silicates, having a Na₂O:SiO₂ molar ratio of from 1:2 to 1:2.8. Preferably used as crystalline silicates, which may be present alone or in a mixture with amorphous silicates, are crystalline phyllosilicates of general formula Na₂SiₓO₂ₓ₊₁.yH₂O, where x, referred to as the module, is a number from 1.9 to 4, y is a number from 0 to 20, and preferred values for x are 2, 3 or 4. Non-limiting crystalline phyllosilicates are those in which x in the stated general formula attains the values 2 or 3. In particular, both beta-sodium and delta-sodium disilicates (Na₂Si₂O₅.yH₂O) are preferred. Practically water-free crystalline alkali silicates of the above general formula, in which x is a number from 1.9 to 2.1 and which are produced from amorphous alkali silicates, may also be used in agents. In a further non-limiting embodiment of agents, a crystalline sodium phyllosilicate having a module of 2 to 3, as can be produced from sand and soda, is used. Crystalline sodium silicates having a module in the range of from 1.9 to 3.5 are used in a further non-limiting embodiment of agents. If alkali aluminosilicate, in particular zeolite, is also present as an additional builder, the weight ratio of aluminosilicate to silicate, in each case based on water-free active substances, is from 1:10 to 10:1. In agents containing both amorphous and crystalline alkali silicates, the weight ratio of amorphous alkali silicate to crystalline alkali silicate is from 1:2 to 2:1 and in particular from 1:1 to 2:1.

Builders are, if desired, contained in the agents in amounts of up to 60 wt. %, in particular from 5 wt. % to 40 wt. %. Laundry aftertreatment agents, for example softeners, are free of inorganic builder.

In particular organic peracids or peracid salts of organic acids, such as phthalimidopercapronic acid, perbenzoic acid or salts of diperdodecanedioic acid, hydrogen peroxide and inorganic salts that release hydrogen peroxide under the application conditions, such as perborate, percarbonate and/or persilicate, are suitable as peroxygen compounds. If solid peroxygen compounds are intended to be used, these may be used in the form of powders or granules, which may also be coated in a manner known in principle. The optional use of alkali percarbonate, alkali perborate monohydrate or, in particular in liquid agents, hydrogen peroxide in the form of aqueous solutions containing from 3 wt. % to 10 wt. % hydrogen peroxide. If an agent contains bleaching agents, such as peroxygen compounds, these are present in amounts of up to 50 wt. %, in particular from 5 wt. % to 30 wt. %. The addition of small amounts of known bleaching agent stabilizers such as phosphonates, borates or metaborates, metasilicates, and magnesium salts such as magnesium sulfate, may be expedient.

Compounds which, under perhydrolysis conditions, result in aliphatic peroxocarboxylic acids having 1 to 10 carbon atoms, in particular 2 to 4 carbon atoms, and/or optionally substituted perbenzoic acid, may be used as bleach activators. Substances that have O-acyl and/or N-acyl groups of the stated number of C atoms and/or optionally substituted benzoyl groups are suitable. Non-limiting examples are polyacylated alkylene diamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoyl succinimide (NOSI), acylated phenolsulfonates, in particular n-nonanoyl- or isononanoyloxybenzenesulfonate (n- or iso-NOBS), carboxylic acid anhydrides, in particular phthalic acid anhydride, acylated polyhydric alcohols, in particular triacetin, ethylene glycol diacetate, 2,5-diacetoxy-2,5-dihydrofuran and enol ester, and acetylated sorbitol and mannitol or mixtures thereof (SORMAN), acylated sugar derivatives, in particular pentaacetyl glucose (PAG), pentaacetyl fructose, tetraacetyl xylose and octaacetyl lactose, and acetylated, optionally N-alkylated glucamine and gluconolactone, and/or N-acylated lactams, for example N-benzoylcaprolactam. Hydrophilically substituted acyl acetals and acyl lactams are likewise used. Combinations of conventional bleach activators may also be used. Bleach activators of this kind may be contained in a typical amount range, in amounts of from 1 wt. % to 10 wt. %, in particular from 2 wt. % to 8 wt. %, based on the overall agent.

In addition to or instead of the conventional bleach activators listed above, sulfonimines and/or bleach-enhancing transition metal salts or transition metal complexes may also be contained as what are referred to as bleach catalysts.

Suitable as enzymes that can be used in the agents are those from the class of proteases, cutinases, amylases, pullulanases, hemicellulases, cellulases, lipases, oxidases and peroxidases, and mixtures thereof. Enzymatic active ingredients obtained from fungi or bacteria, such as Bacillus subtilis, Bacillus licheniformis, Streptomyces griseus, Humicola lanuginosa, Humicola insolens, Pseudomonas pseudoalcaligenes or Pseudomonas cepacia are particularly suitable. The optionally used enzymes may be adsorbed on carrier substances and/or embedded in coating substances to protect said enzymes from premature inactivation. The enzymes are, if desired, contained in the agents in amounts no greater than 5 wt. %, in particular from 0.2 wt. % to 2 wt. %.

The agents may optionally contain, for example, derivatives of diaminostilbene disulfonic acid or alkali metal salts thereof as optical brighteners. Suitable are, for example, salts of 4,4′-bis(2-anilino-4-morpholino-1,3,5-triazinyl-6-amino)stilbene-2,2′-disulfonic acid or compounds having a similar structure which, instead of the morpholino group, have a diethanolamino group, a methylamino group, an anilino group or a 2-methoxyethylamino group.

Suitable foam inhibitors include, for example, organopolysiloxanes and mixtures thereof with microfine, optionally silanated silicic acid and paraffin waxes and mixtures thereof with silanated silicic acid or bis-fatty acid alkylene diamides. Mixtures of various foam inhibitors are also advantageously used, for example those made up of silicones, paraffins or waxes. The foam inhibitors, in particular silicone-containing and/or paraffin-containing foam inhibitors, are bound to a granular carrier substance that is soluble or dispersible in water. Mixtures of paraffin waxes and bistearylethylenediamides are particularly preferred.

In addition, the agents may also contain components that positively influence the capability for washing out oil and grease from textiles, or what are referred to as soil-release active ingredients. This effect is particularly apparent when a textile is soiled which has been previously washed several times using an agent that contains this deoiling and degreasing component. Non-limiting deoiling and degreasing components include, for example, nonionic cellulose ethers such as methylcellulose and methylhydroxypropylcellulose having a proportion of from 15 to 30 wt. % methoxyl groups and from 1 to 15 wt. % hydroxypropoxyl groups, in each case based on the nonionic cellulose ether, and the polymers of phthalic acid and/or terephthalic acid, or derivatives thereof, with monomeric and/or polymeric diols, in particular polymers of ethylene terephthalates and/or polyethylene glycol terephthalates or anionically and/or nonionically modified derivatives thereof.

The agents may also contain dye transfer inhibitors, in amounts of from 0.1 wt. % to 2 wt. %, in particular from 0.1 wt. % to 1 wt. %, which, in a embodiment, are polymers of vinylpyrrolidone, vinyl imidazole or vinyl pyridine-N-oxide, or copolymers thereof.

The function of graying inhibitors is to keep the dirt that is removed from the textile fiber suspended in the liquor. Water-soluble colloids, which are usually organic, are suitable for this purpose, for example starch, sizing material, gelatine, salts of ethercarboxylic acids or ethersulfonic acids of starch or of cellulose, or salts of acidic sulfuric acid esters of cellulose or of starch. Water-soluble polyamides containing acidic groups are also suitable for this purpose. Starch derivatives other than those mentioned above may also be used, for example aldehyde starches. Cellulose ethers, such as carboxymethylcellulose (Na salt), methylcellulose, hydroxyalkylcellulose, and mixed ethers, such as methylhydroxyethylcellulose, methylhydroxypropylcellulose, methylcarboxymethylcellulose and mixtures thereof, may be used, for example, in amounts of from 0.1 to 5 wt. %, based on the agents.

The organic solvents that are usable in the agents, in particular when the agents are present in liquid or paste-like form, include alcohols having 1 to 4 carbon atoms, in particular methanol, ethanol, isopropanol and tert-butanol, diols having 2 to 4 carbon atoms, in particular ethylene glycol and propylene glycol, and mixtures thereof, and the ethers that are derivable from the mentioned compound classes. Water-miscible solvents of this kind are present in the agents in amounts no greater than 30 wt. %, in particular from 6 wt. % to 20 wt. %.

For setting a desired pH that does not result from mixing the other components themselves, the agents may contain acids that are compatible with the system and the environment, in particular citric acid, acetic acid, tartaric acid, malic acid, lactic acid, glycolic acid, succinic acid, glutaric acid and/or adipic acid, but also mineral acids, in particular sulfuric acid, or bases, in particular ammonium or alkali hydroxides. pH regulators of this kind are optionally contained in the agents in amounts no greater than 20 wt. %, in particular from 1.2 wt. % to 17 wt. %.

The production of solid agents, i.e. in particular washing or cleaning agents, poses no difficulties, and may take place in a manner known in principle, for example by spray drying or granulation, with an optional peroxygen compound and an optional bleach catalyst being optionally added later. For the production of agents having an increased bulk weight, in particular in the range of from 650 g/l to 950 g/l, a method having an extrusion step is used. The production of liquid agents does not pose any difficulties either, and may likewise take place in a known manner.

According to a non-limiting embodiment, the agent may be used to significantly reduce the perfume proportion in washing, cleaning and body care agents. It is thus possible to also provide perfumed products for particularly sensitive consumers who, due to specific intolerances and irritations, can use the normally perfumed products only on a limited basis or not at all.

In various embodiments, the washing or cleaning agents are present in liquid or in solid form.

A non-limiting solid, in particular a powdered, washing agent, in addition to the compound, may also contain in particular components that are selected from the following, for example:
anionic surfactants, such as alkylbenzenesulfonate, alkyl sulfate, for example in amounts from 5 to 30 wt. %,
nonionic surfactants, such as fatty alcohol polyglycol ethers, alkyl polyglucoside, fatty acid glucamide, for example in amounts from 0.5 to 15 wt. %,
builders, for example zeolite, polycarboxylate, sodium citrate, in amounts of, for example, from 0 to 70 wt. %, alternatively from 5 to 60 wt. %, alternatively from 10 to 55 wt. %, in particular from 15 to 40 wt. %,
alkalis, such as sodium carbonate, in amounts of, for example, from 0 to 35 wt. %, advantageously from 1 to 30 wt. %, alternatively from 2 to 25 wt. %, in particular from 5 to 20 wt. %,
bleaching agents, such as sodium perborate, sodium percarbonate, in amounts of, for example, from 0 to 30 wt. %, advantageously from 5 to 25 wt. %, alternatively from 10 to 20 wt. %,
corrosion inhibitors, for example sodium silicate, in amounts of, for example, from 0 to 10 wt. %, advantageously from 1 to 6 wt. %, alternatively from 2 to 5 wt. %, in particular from 3 to 4 wt. %,
stabilizers, for example phosphonates, advantageously from 0 to 1 wt. %,
foam inhibitors, for example soap, silicone oils, paraffins, advantageously from 0 to 4 wt. %, alternatively from 0.1 to 3 wt. %, in particular from 0.2 to 1 wt. %,
enzymes, for example proteases, amylases, cellulases, lipases, advantageously from 0 to 2 wt. %, alternatively from 0.2 to 1 wt. %, in particular from 0.3 to 0.8 wt. %,
graying inhibitors, for example carboxymethylcellulose, advantageously from 0 to 1 wt. %,
discoloration inhibitors, for example polyvinylpyrrolidone derivatives, alternatively from 0 to 2 wt. %,
adjusters, for example sodium sulfate, advantageously from 0 to 20 wt. %,
optical brighteners, for example stilbene derivatives, biphenyl derivatives, advantageously from 0 to 0.4 wt. %, in particular from 0.1 to 0.3 wt. %,
optionally further fragrances,
optionally water,
optionally soap,
optionally bleach activators,
optionally cellulose derivatives,
optionally soil-repellent agents,
in wt. %, in each case based on the overall agent.

In another non-limiting embodiment, the agent is present in liquid form, such as in gel form. Preferred liquid washing or cleaning agents and cosmetics have water contents of, for example, from 10 to 95 wt. %, alternatively from 20 to 80 wt. %, and in particular from 30 to 70 wt. %, based on the overall agent. In the case of liquid concentrates, the water content may also be particularly low, for example <30 wt. %, alternatively <20 wt. %, in particular <15 wt. %, in wt. % in each case based on the overall agent. The liquid agents may also contain nonaqueous solvents.

A liquid, in particular gel-form, washing agent, in addition to the compound, may also contain in particular components that are selected from the following, for example:
anionic surfactants, such as alkylbenzenesulfonate, alkyl sulfate, for example in amounts of from 5 to 40 wt. %,
nonionic surfactants, such as fatty alcohol polyglycol ethers, alkyl polyglucoside, fatty acid glucamide, for example in amounts of from 0.5 to 25 wt. %,
builders, for example zeolite, polycarboxylate, sodium citrate, advantageously from 0 to 15 wt. %, alternatively from 0.01 to 10 wt. %, in particular from 0.1 to 5 wt. %,
foam inhibitors, for example soap, silicone oils, paraffins, in amounts of, for example, from 0 to 10 wt. %, advantageously from 0.1 to 4 wt. %, alternatively from 0.2 to 2 wt. %, in particular from 1 to 3 wt. %,
enzymes, for example proteases, amylases, cellulases, lipases, in amounts of, for example, from 0 to 3 wt. %, advantageously from 0.1 to 2 wt. %, alternatively from 0.2 to 1 wt. %, in particular from 0.3 to 0.8 wt. %,
optical brighteners, for example stilbene derivatives, biphenyl derivatives, in amounts of, for example, from 0 to 1 wt. %, advantageously from 0.1 to 0.3 wt. %, in particular from 0.1 to 0.4 wt. %,
optionally further fragrances,
optionally stabilizers,
water,
optionally soap, in amounts of, for example, from 0 to 25 wt. %, advantageously from 1 to 20 wt. %, alternatively from 2 to 15 wt. %, in particular from 5 to 10 wt. %,
optionally solvents (e.g. alcohols), advantageously from 0 to 25 wt. %, alternatively from 1 to 20 wt. %, in particular from 2 to 15 wt. %,
in wt. %, in each case based on the overall agent.

A liquid softener, in addition to the ketone, may also contain in particular components that are selected from the following:
cationic surfactants, such as in particular esterquats, for example in amounts of from 5 to 30 wt. %,
cosurfactants, for example glycerol monostearate, stearic acid, fatty alcohols, fatty alcohol ethoxylates, for example in amounts of from 0 to 5 wt. %, alternatively from 0.1 to 4 wt. %,
emulsifiers, for example fatty amine ethoxylates, for example in amounts of from 0 to 4 wt. %, alternatively from 0.1 to 3 wt. %,
optionally further fragrances,
dyes, alternatively in the ppm range,
stabilizers, alternatively in the ppm range,
solvents, for example water, in amounts of alternatively from 60 to 90 wt. %,
   in wt. %, in each case based on the overall agent.

A further aspect relates to a method for fragrancing surfaces, in which method the surface to be fragranced is brought into contact with the pro-fragrance of general formula (I), and the surface is subsequently exposed to light.

Within the scope, all the above-mentioned embodiments or the features described in each case may also be individually combined. Furthermore, the term “comprising” also covers the alternative in which the products/methods/uses in relation to which the term “comprising” is used consists only of the subsequently described elements.

### Practical Examples

### Preparation of (4R,7R)-4-methyl-7-(prop-1-en-2-yl)-1,2-diazaspiro[2.5]oct-1-ene

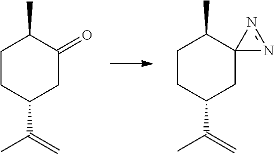

5.0 ml (30.6 mmol, 1.0 eq) of dihydrocarvone was dropped into 44 ml (306 mmol, 10 eq) of methanolic ammonia solution (c=7.0 mol/1) at 0° C. and stirred for two hours at this temperature. The temperature was then reduced to −18° C., and 3.5 g (76.4 mmol, 2.5 eq) of hydroxylamine sulfonic acid was added in portions. The cooling bath was left to thaw and was stirred for 20 hours at room temperature. Subsequently, the solvent and excess ammonia were removed in vacuo, the intermediate product was dissolved in 153 ml of acetone (c=0.2 mol/1), and 4.6 g (45.8 mmol, 1.5 eq) of chromium trioxide in 66 ml of sulfuric acid (c(H₂SO₄)=3.8 mol/l, c(CrO₃)=0.7 mol/1) was dropped in within 45 minutes. After four hours of stirring, 150 ml of water was added, extraction was carried out three times using 150 ml of dichloromethane, washing was carried out using 150 ml of saturated sodium chloride solution, drying was carried out using magnesium sulfate, and the solvent was removed in vacuo. The raw product was purified by column chromatography on silica gel using cyclohexane/ethyl acetate (9:1).

Yield: 1.8 g (11.0 mmol, 36%)

M=(C₁₀H₁₆N₂)=164.25 g/mol

Rf=0.67

¹H-NMR (CDCl₃, 100 MHz): δ (ppm)=4.59 (m, 1H), 4.56 (s, 1H), 1.11 (tt, J=12.0, 3.3 Hz), 1.92-1.64 (m, 4H), 1.58 (s, 3H), 1.38-1.20 (m, 2H on C-6), 0.37 (ddd, J=13.8, 3.8, 2.1 Hz, 1H), 0.11 (d, J=7.1 Hz, 3H).

¹³C-NMR (CDCl₃, 100 MHz): δ (ppm)=148.3, 109.1, 43.4, 37.5, 33.9, 33.0, 31.4, 31.0, 20.7, 17.1.

MS (EtOAc, El): τ_{R}=8.18 min; m/z [%]=93.0, 91.0, 79.0, 77.0, 67.0.

### Exposure to Light of (4R,7R)-4-methyl-7-(prop-1-en-2-yl)-1,2-diazaspiro[2.5]oct-1-ene

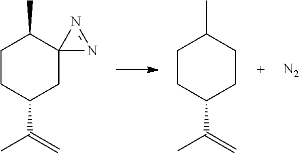

16.4 mg (0.10 mmol) of (4R,7R)-4-methyl-7-(prop-1-en-2-yl)-1,2-diazaspiro[2.5]oct-1-ene was dissolved in 10 ml of methanol. After one hour of photolysis at a wavelength of A=350 nm, limonene was obtained as clearly the main cleavage product, in addition to approximately 10% isolimonene. After three hours of exposure to light, no more diazirine could be detected.

Detection took place by means of a GC (gas chromatography) comparison with the standard substances limonene and isolimonene (from Aldrich).

What is claimed is:

1. A compound of formula (I) 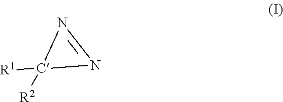
wherein R¹ and/or R² each have, independently of one another, an abstractable H atom in the alpha position in relation to C′.

2. The compound according to claim 1, wherein R¹ and R² are each selected, independently of one another, from
a linear, aliphatic, olefinic or open-chain organic group having from 2 to 20 carbon atoms, and having from 0 to 10 heteroatoms selected from N, O, S and Si; or
a branched or cyclic organic group having from 3 to 20 carbon atoms, and having from 0 to 10 heteroatoms selected from N, O, S and Si; or
an aromatic or heteroaromatic organic group having from 4 to 20 carbon atoms, and having from 0 to 10 heteroatoms selected from N, O, S and Si; or
R¹ and R² are interconnected by a bridge Q for ring closure and form a bridging part —R¹-Q-R²—, wherein R¹ and R² each representing, independently of one another, a group having from 1 to 5 carbon atoms; and Q representing a substituted or unsubstituted bridging group having from 1 or 2 to 20 carbon atoms, and having from 0 to 10 heteroatoms selected from N, O, S and Si.

3. The compound according to claim 1, wherein R¹ and/or R² are each, independently of one another,
a. a group of formula —CH(OH)—R³, —CH₂—C(O)—R³, aryl, heteroaryl, CH₂ aryl or CH₂ heteroaryl, wherein R³ is selected from the group consisting of hydrogen, substituted or unsubstituted, linear or branched alkyl, alkenyl, or alkynyl having up to 20 carbon atoms, substituted or unsubstituted, linear or branched heteroalkyl, heteroalkenyl or heteroalkynyl having up to 20 carbon atoms, and having from 1 to 6 heteroatoms selected from O, S and N, substituted or unsubstituted aryl having up to 20 carbon atoms, substituted or unsubstituted heteroaryl having up to 20 carbon atoms, and having from 1 to 6 heteroatoms selected from O, S and N, cycloalkyl or cycloalkenyl having up to 20 carbon atoms, and heterocycloalkyl or heterocycloalkenyl having up to 20 carbon atoms, and having from 1 to 6 heteroatoms selected from O, S and N; or
b. a substituted or unsubstituted, linear alkyl having from 1 to 5 carbon atoms, or a substituted or unsubstituted, branched alkyl having from 3 to 5 carbon atoms.

4. The compound according to claim 2, wherein the bridging part —R¹-Q-R²— is a hydrocarbon.

5. The compound according to claim 1, wherein the compound has the following formula (III)

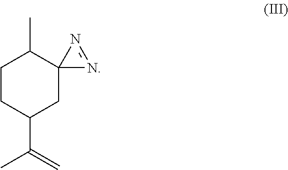

6. The compound according to claim 1, wherein R¹ or R² or the bridging part —R¹-Q-R²— is a group derived from a fragrance alkene.

7. The compound according to claim 6, wherein the bridging alkene is limonene.

8. A method for producing a compound of formula (I) 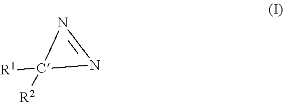
wherein R¹ and/or R² each have, independently of one another, an abstractable H atom in the alpha position in relation to C′, by dehydration of a diaziridine of general formula (IV)

9. A washing or cleaning agent, containing at least one compound from claim 1.

10. The washing or cleaning agent of claim 9, wherein the compound comprises at least one of:
a. an amount of the at least one compound ranging from 0.001 to 5 wt. % based on the overall washing or cleaning agent,
b. at least one surfactant selected from the group consisting of an anionic surfactant, a cationic surfactant, a nonionic surfactant, a zwitterionic surfactant, an amphoteric surfactant, and mixtures thereof,
c. the compound is present in liquid or solid form; and
d. combinations thereof.

11. An air-care agent containing at least one compound from claim 1, wherein the amount of the compound ranges from about 0.001 to about 5 wt. % based on the overall air-care agent.

12. A cosmetic agent containing at least one compound according to claim 1, wherein the compound ranges from about 0.001 to about 5 wt. % based on the overall cosmetic agent.

13. A method for fragrancing surfaces comprising:
applying at least one compound from claim 1 is to at least one surface to be fragranced; and
exposing the at least on surface to light.
